(19) United States
(12) Patent Application Publication
(10) Pub. No.: US 2026/0306369 A1
(43) Pub. Date: Oct. 8, 2026

(54) AEROSOL PRODUCT

(71) Applicant: OSAKA PHARMACEUTICAL CO., LTD., Osaka (JP)

(72) Inventors: Chika SHIBATA, Osaka (JP); Hitomi FUKUDA, Osaka (JP)

(21) Appl. No.: 19/478,666

(22) PCT Filed: Apr. 26, 2024
(86) PCT No.: PCT/JP2024/016376

§ 371 Date: Oct. 24, 2025

(30) Foreign Application Priority Data
Apr. 28, 2023 (JP) 2023-074244
Nov. 2, 2023 (JP) 2023-188721

(51) Int. Cl. A61K 8/04; A45D 34/04; A61Q 5/02; A61Q 19/10

(52) U.S. Cl. CPC A61K 8/046; A45D 34/04; A61Q 5/02; A61Q 19/10; A45D 2200/057; A61K 2800/222; A61K 2800/413; A61K 2800/87

(57) ABSTRACT

An object of the present invention is to provide a product that is excellent in portability, contains bubbles when a liquid composition is applied to the skin, and offers a favorable feeling in use. The aerosol product comprises a container 1, a cap portion 7 provided with a nozzle, a liquid composition 2 contained in the container 1, and a non-flammable compressed gas G filled in the container 1 together with the liquid composition 2, wherein a diameter h of a discharge portion of the nozzle and a nozzle length L satisfy L/h=1 to 20.

### TECHNICAL FIELD

The present invention relates to an aerosol product for applying a liquid composition contained within a container. More specifically, the invention pertains to an aerosol product which, when applied to the skin, contains bubbles having diameters on the nanometer scale, thereby promoting penetration of the liquid composition into the skin or hair, or providing excellent cleansing properties.

### BACKGROUND ART

Conventionally, cosmetic compositions for use on human skin that contain bubbles with diameters on the nanometer scale have been known.

For example, Patent Document 1 discloses a cosmetic composition that provide a favorable feel of use, being well accepted by the skin and not leaving a sticky sensation even after application. The cosmetic composition contains a nanobubble liquid and other cosmetic ingredients, wherein the nanobubble liquid contains oil and bubbles, and the bubbles have diameters ranging from 5 nm to 500 nm.

### PRIOR ART DOCUMENT

### Patent Documents

Patent Document 1: Japanese Laid-Open Patent Publication No. 2022-103130

### SUMMARY OF THE INVENTION

### Problems to be Solved by the Invention

There is still a demand for a cosmetic composition that has a simple configuration, is excellent in portability, contains bubbles when actually applied to the skin, and provides a good sensory characteristics upon use.

Accordingly, it is an object of the present invention to provide a product that is excellent in portability and contains bubbles when used on the body of a human or the like.

### Means for Solving the Problems

That is, the present invention relates to an aerosol product comprising: a container and a cap provided with a nozzle; a liquid composition contained in the container; and a non-flammable compressed gas filled in the container together with the liquid composition.

In a preferred embodiment, the aerosol product may be as follows.

[1]

An aerosol product comprising: a container and a cap provided with a nozzle; a liquid composition contained in the container; and a non-flammable compressed gas filled in the container together with the liquid composition, wherein the diameter h of a discharge portion of the nozzle and the nozzle length L satisfy the relationship L/h=1 to 20.

[2]

The aerosol product according to item [1], wherein the non-flammable compressed gas is sealed at a pressure of 0.3 to 1.0 MPa at 25° C.

[3]

The aerosol product according to item [1] or [2], wherein the viscosity of the liquid composition is 0 to 100,000 mPa·s at 25° C.

[4]

The aerosol product according to any one of items [1] to [3], wherein the liquid composition is selected from the group consisting of a solubilized composition, a gel, and an emulsion.

[5]

The aerosol product according to any one of items [1] to [4], wherein the liquid composition comprises water or a polyhydric alcohol.

[6]

The aerosol product according to any one of items [1] to [5], wherein the liquid composition comprises at least one selected from the group consisting of glycyrrhizic acid or a salt thereof, glycyrrhetinic acid or a salt thereof, allantoin or a salt thereof, vitamin C group and derivatives thereof, tranexamic acid, arbutin, placenta, vitamin B group and derivatives thereof, vitamin E group and derivatives thereof, vitamin A group and derivatives thereof, urea, and polysaccharides.

[7]

The aerosol product according to any one of items [1] to [6], wherein the non-flammable compressed gas is nitrogen.

[8]

The aerosol product according to any one of items [1] to [7], wherein the liquid composition is a topical skin preparation, an oral care product, a cleansing product, or a hair-related product.

### Effect of the Invention

According to the aerosol product of the present invention, it can be carried and transported, and is therefore excellent in portability.

Furthermore, when applied to the skin, it can contain a large number of bubbles having diameters on the nanometer scale.

### BRIEF DESCRIPTION OF THE DRAWINGS

FIG. 1 is a central cross-sectional view of an aerosol product according to one embodiment of the present invention.

FIG. 2 is a side view of a container cap in the aerosol product according to one embodiment of the present invention.

FIG. 3 is a top view of a container cap in the aerosol product according to one embodiment of the present invention.

FIG. 4 is a side view of a container cap in the aerosol product according to one embodiment of the present invention.

FIG. 5 is a top view of a container cap in the aerosol product according to one embodiment of the present invention.

FIG. 6 is a side view of a container cap in the aerosol product according to one embodiment of the present invention.

FIG. 7 is a top view of a container cap in the aerosol product according to one embodiment of the present invention.

FIG. 8 is a side view of a container cap in the aerosol product according to one embodiment of the present invention.

FIG. 9 is a top view of a container cap in the aerosol product according to one embodiment of the present invention.

FIG. 10 is a cross-sectional view of a portion of a container cap in the aerosol product according to one embodiment of the present invention.

### MODE FOR CARRYING OUT THE INVENTION

Hereinafter, embodiments of the aerosol product of the present invention will be described in detail.

### [Aerosol Product]

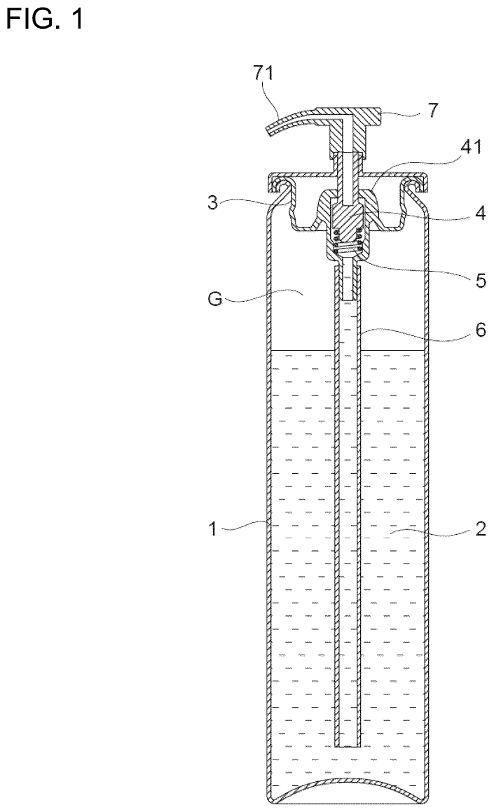

An aerosol product according to one non-limiting embodiment of the present invention is shown in FIG. 1. A container 1 in the aerosol product of the present invention is a member that contains a liquid composition 2, a non-flammable compressed gas G, and the like therein. Since the interior of the container 1 is filled with the non-flammable compressed gas G, the container has pressure resistance so as not to rupture under a predetermined pressure, and may be made of a metal such as an aluminum alloy or tin, or a synthetic resin having a predetermined thickness. In this embodiment, the container 1 has a hollow cylindrical body having a closed bottom and having an opening at its upper portion. As shown in FIG. 1, the opening at the upper portion is sealed with a mounting cup 3 provided with a stem 4 and a cap portion 7. The stem 4 and a spring 5 are fixed to the mounting cup 3 by a housing. The cap portion 7 is provided with a pump portion having a nozzle portion. In this embodiment, the liquid composition 2 and the non-flammable compressed gas G are contained so as to be in contact with the inner surface of the container 1. In other embodiments, however, another container or the like may be enclosed inside the container 1, and the liquid composition 2 may be contained inside such other container or the like.

### (Liquid Composition)

The liquid composition contained in the aerosol product of the present invention is a liquid composition contained in the container, and more specifically, is in a liquid state at room temperature of 20 to 30° C. Preferably, it contains water or a polyhydric alcohol.

The water that may be contained in the liquid composition is preferably water that meets the standards of the Japanese Pharmacopoeia. Examples include ordinary water such as tap water or well water; purified water obtained by treating ordinary water through one or more of distillation, ion exchange treatment using an ion exchange membrane, or ultrafiltration treatment using an ultrafiltration membrane; and sterilized purified water obtained by sterilizing purified water by heating or the like. The content ratio of water in the liquid composition is preferably about 0.1 to 98% by mass, more preferably 1 to 98% by mass. The amount of water may also be 30 to 98% by mass, 40 to 95% by mass, or 50 to 90% by mass. In certain embodiments, it is particularly preferred that the water content be 60 to 85% by mass.

The polyhydric alcohol that may be contained in the liquid composition is not particularly limited, but may be a polyhydric alcohol having two or more hydroxyl groups, such as glycerin, dipropylene glycol, propylene glycol, 1,3-propanediol, butylene glycol, sorbitol, or polyethylene glycol. The content ratio of the polyhydric alcohol in the liquid composition is preferably about 0.1 to 60% by mass, more preferably 0.5 to 40% by mass. The amount of the polyhydric alcohol may also be 0.5 to 30% by mass, 0.5 to 20% by mass, or 0.5 to 15% by mass. In certain embodiments, it is particularly preferred that the content be 1 to 15% by mass.

The liquid composition may optionally contain a polysaccharide. The polysaccharide contained may function as a moisturizing ingredient. Here, a polysaccharide refers to a compound in which multiple monosaccharides are linked by glycosidic bonds. Examples of polysaccharides usable in the present invention include at least one selected from the group consisting of heparinoid, hyaluronic acid, and sodium hyaluronate. These polysaccharides may be used alone or in combination of two or more.

The content ratio of the polysaccharide in the liquid composition is preferably 0.0001 to 5% by mass, more preferably 0.01 to 4% by mass, and most preferably 0.1 to 3% by mass. When the content ratio of the polysaccharide moisturizing ingredient falls within this range, the composition, when applied to the human body or the like in the form of an applicate containing numerous bubbles having diameters of 1 nm or more and less than 1000 nm, penetrates the skin to enhance moisturization and facilitates the sustained retention of moisture.

(pH)

From the viewpoints of stability and safety of the components, it is preferred that the liquid composition of the present invention have a pH of 2 to 9.

More preferably, the pH is 3 to 8, and still more preferably 4 to 7. The pH of the liquid composition can be measured at 20° C. using a commercially available pH meter (for example, model F-52® manufactured by Horiba, Ltd.).

### (Viscosity)

From the viewpoint of component stability and the like, the viscosity of the liquid composition of the present invention is preferably about 0 to 100,000 mPa·s, more preferably about 0.5 to 80,000 mPa·s, and still more preferably about 3 to 60,000 mPa·s. The viscosity is measured at 25° C. using SPINDLE No. M4, at a rotational speed of 12 rpm for 3 minutes. However, in cases where the viscosity is 50,000 mPa·s or more, the measurement is conducted at a rotational speed of 6 rpm.

If measurement is not possible under the above conditions, the measurement may be performed by appropriately changing the conditions. The viscosity of the liquid composition can be measured at 25° C. using a commercially available viscometer (for example, model TVB-10M manufactured by Tokki Sangyo Co., Ltd.).

The liquid composition of the present invention may further contain, for example, pharmacologically active ingredients, moisturizing ingredients other than polysaccharides, oils, surfactants, thickeners, and the like.

Examples of pharmacologically active ingredients include: anti-inflammatory ingredients such as glycyrrhizic acid or a salt thereof, glycyrrhetinic acid or a salt thereof, and allantoin or a salt thereof; whitening ingredients such as vitamin C group and derivatives thereof (e.g., ascorbic acid, 3-O-ethyl ascorbic acid), tranexamic acid, arbutin, and placenta; as well as vitamin B group and derivatives thereof (e.g., riboflavin, niacinamide), vitamin E group and derivatives thereof, vitamin A group and derivatives thereof, and urea. These pharmacologically active ingredients may be used alone or in combination of two or more.

The content ratio of these pharmacologically active ingredients in the liquid composition is not particularly limited. For example, the content ratio of glycyrrhetinic acid, glycyrrhizic acid, or a salt thereof in the liquid composition is preferably 0.001 to 5.0% by mass, more preferably 0.05 to 3.0% by mass. The content ratio of allantoin or a salt thereof in the liquid composition is preferably 0.001 to 5.0% by mass, more preferably 0.05 to 3.0% by mass. The amount of vitamin C group and derivatives thereof is preferably 0.1 to 10% by mass, more preferably 0.3 to 5% by mass. The amount of vitamin E group is preferably 0.01 to 5% by mass, more preferably 0.02 to 3% by mass. The amount of vitamin A group is preferably 0.0005 to 0.5% by mass, more preferably 0.0007 to 0.2% by mass. The amount of urea is preferably 0.1 to 30% by mass, more preferably 1 to 20% by mass. When the content ratio of these pharmacologically active ingredients is within these ranges, upon application to the skin of a human or the like, the composition can penetrate the skin and impart various medicinal effects.

Examples of ingredients other than polysaccharides that may be contained in the liquid composition include ceramides and plant extracts. These function as moisturizing ingredients and may be used alone or in combination of two or more. By using these moisturizing ingredients other than polysaccharides together with polysaccharide moisturizing ingredients, the moisturizing effect can be enhanced, and the skin can be imparted with firmness and elasticity.

The content ratio of these ingredients in the liquid composition is not particularly limited. For example, the content ratio of ceramides and/or plant extracts in the liquid composition is preferably 0.001 to 5.0% by mass, more preferably 0.05 to 3.0% by mass.

When the liquid composition is a formulation intended for moisturizing, preferred examples of oils that may be contained include hydrocarbons such as squalane, vegetable oils such as olive oil, silicone oils such as dimethylpolysiloxane, fatty alcohols having 14 to 22 carbon atoms such as stearyl alcohol and behenyl alcohol, and ester oils such as triethylhexanoin. These oils may be used alone or in combination of two or more. By means of these oils, for example, an emulsion that is an oil-in-water type or a water-in-oil type can be prepared, which suppresses evaporation of water from the skin to which the applicate is applied, thereby enhancing the moisturizing effect and imparting firmness and elasticity to the skin.

When the liquid composition is a formulation intended for cleansing, preferred examples of oils that may be contained include ester oils such as triethylhexanoin, cetyl ethylhexanoate, and isopropyl palmitate; hydrocarbons such as liquid paraffin and light liquid isoparaffin; and vegetable oils such as olive oil and macadamia nut oil. These oils may be used alone or in combination of two or more. By means of these oils, cleansing power can be improved, rinsing can be made easier, and an excellent feeling of use can be obtained.

When the liquid composition is a formulation intended for moisturizing, the content ratio of the oil in the liquid composition 2 is preferably 0.05 to 15% by mass, more preferably 0.5 to 10% by mass, and particularly preferably 0.5 to 7% by mass. When the content ratio of the oil is within this range, as described above, evaporation of water from the skin to which the applicate is applied can be suppressed, thereby enhancing the moisturizing effect and imparting firmness and elasticity to the skin.

When the liquid composition is a formulation intended for cleansing, the content ratio of the oil is preferably 10 to 98% by mass, more preferably 15 to 95% by mass, and particularly preferably 20 to 90% by mass. When the content ratio of the oil is within this range, as described above, cleansing power can be improved, rinsing can be made easier, and an excellent feeling of use can be obtained.

Examples of surfactants that may be contained in the liquid composition include nonionic surfactants, anionic surfactants, cationic surfactants, and amphoteric surfactants.

When the liquid composition is a formulation intended for moisturizing, preferred examples of nonionic surfactants include polyoxyethylene alkyl ethers such as polyoxyethylene cetyl ether and polyoxyethylene stearyl ether; polyoxyethylene fatty acid esters such as polyoxyethylene glycol monostearate; glycerin fatty acid esters such as glyceryl monostearate and sorbitan fatty acid esters; polyhydric alcohol fatty acid esters such as sucrose fatty acid esters; fatty acid esters of polyoxyethylene-added polyhydric alcohols; and fatty acid diethanolamides. Specific examples include polyoxyethylene sorbitan tetraoleate, polyoxyethylene (20) sorbitan oleate, lauric acid diethanolamide, and coconut fatty acid (C8-18) diethanolamide.

Preferred examples of anionic surfactants include:
Carboxylic acid type anionic surfactants such as sodium laurate, sodium stearate, sodium laureth-6 carboxylate (polyoxyethylene (4.5) lauryl ether acetate sodium salt), sodium lauroyl sarcosinate, sodium octanoate, sodium decanoate, sodium myristate, sodium palmitate, and sodium coconut fatty acid (C8-18) sarcosinate;
Sulfonic acid type anionic surfactants such as sodium laurylsulfoacetate, sodium 1-hexanesulfonate, sodium 1-octanesulfonate, sodium 1-decanesulfonate, sodium 1-dodecanesulfonate, sodium toluenesulfonate, sodium cumenesulfonate, sodium naphthalenesulfonate, disodium naphthalenedisulfonate, trisodium naphthalenetrisulfonate, and sodium alpha-olefinsulfonate;
Sulfate ester type anionic surfactants such as sodium lauryl sulfate, sodium myristyl sulfate, sodium laureth sulfate (polyoxyethylene (3) lauryl ether sulfate sodium salt), sodium cetyl sulfate, sodium cocoglyceride sulfate (hydrogenated coconut fatty acid glyceride sulfate sodium salt), triethanolamine lauryl sulfate, ammonium lauryl sulfate, and triethanolamine laureth sulfate;
Phosphate ester type anionic surfactants such as sodium lauryl phosphate, sodium polyoxyethylene cetyl ether phosphate (polyoxyethylene (5) cetyl ether phosphate sodium salt), lauryl phosphate, and potassium lauryl phosphate.
   Preferred examples of cationic surfactants include:
Quaternary ammonium salt type surfactants such as tetramethylammonium chloride, tetrabutylammonium chloride, dodecyldimethylbenzylammonium chloride, alkyltrimethylammonium chloride, octyltrimethylammonium chloride, dodecyltrimethylammonium chloride, benzalkonium chloride, and benzethonium chloride;
Alkylamine salt type surfactants such as monomethylamine hydrochloride and dimethylamine hydrochloride.
   Preferred examples of amphoteric surfactants include:
Amine oxide type surfactants such as lauryldimethylamine oxide, alkyl (C8-18) dimethylamine oxides (N,N-dimethylalkyl (C8-18) amine oxides), coconut alkyldimethylamine oxide, decyldimethylamine oxide, myristyldimethylamine oxide, dihydroxyethyllaurylamine oxide, and oleyl dimethylamine oxide;
Betaine type surfactants such as lauryldimethylaminoacetic acid betaine, laurylhydroxysulfobetaine, stearyldimethylaminoacetic acid betaine, dodecylaminomethyldimethylsulfopropyl betaine, coconut fatty acid (C8-18) amidopropyl betaine, 2-alkyl-N-carboxymethyl-N-hydroxyethylimidazolinium betaine, and lauramidopropyl betaine.
   These surfactants may be used alone or in combination of two or more.

When the liquid composition is a formulation intended for cleansing, preferred examples of nonionic surfactants include polyoxyethylene sorbitan fatty acid esters such as sorbeth tetraoleate and polyoxyethylene sorbitan coconut fatty acid esters; polyoxyethylene polyhydric alcohol fatty acid esters such as PEG-8 distearate; glycerin fatty acid esters such as glyceryl monostearate and sorbitan fatty acid esters; polyoxyethylene alkyl ethers such as polyoxyethylene cetyl ether; sorbitol fatty acid esters such as polyoxyethylene sorbitol fatty acid esters; polyoxyethylene glycerin fatty acid esters; polyoxyethylene propylene glycol fatty acid esters; polyoxyethylene hydrogenated castor oil and polyoxyethylene hydrogenated castor oil fatty acid esters; alkyl polyglycosides; and polyoxyalkylene-modified silicones such as polyoxyethylene-methylpolysiloxane copolymers.

Among these, polyoxyethylene sorbitan fatty acid esters and polyoxyethylene polyhydric alcohol fatty acid esters are preferred.

Preferred examples of anionic surfactants include:
Carboxylic acid type anionic surfactants such as sodium laurate, sodium stearate, sodium laureth-6 carboxylate (polyoxyethylene (4.5) lauryl ether acetate sodium salt), sodium lauroyl sarcosinate, sodium octanoate, sodium decanoate, sodium myristate, sodium palmitate, and sodium coconut fatty acid (C8-18) sarcosinate;
Sulfonic acid type anionic surfactants such as sodium laurylsulfoacetate, sodium 1-hexanesulfonate, sodium 1-octanesulfonate, sodium 1-decanesulfonate, sodium 1-dodecanesulfonate, sodium toluenesulfonate, sodium cumenesulfonate, sodium naphthalenesulfonate, disodium naphthalenedisulfonate, trisodium naphthalenetrisulfonate, and sodium alpha-olefinsulfonate;
Sulfate ester type anionic surfactants such as sodium lauryl sulfate, sodium myristyl sulfate, sodium laureth sulfate (polyoxyethylene (3) lauryl ether sulfate sodium salt), sodium cetyl sulfate, sodium cocoglyceride sulfate (hydrogenated coconut fatty acid glyceride sulfate sodium salt), triethanolamine lauryl sulfate, ammonium lauryl sulfate, and triethanolamine laureth sulfate;
Phosphate ester type anionic surfactants such as sodium lauryl phosphate, sodium polyoxyethylene cetyl ether phosphate (polyoxyethylene (5) cetyl ether phosphate sodium salt), lauryl phosphate, and potassium lauryl phosphate.
   Preferred examples of cationic surfactants include:
Quaternary ammonium salt type surfactants such as tetramethylammonium chloride, tetrabutylammonium chloride, dodecyldimethylbenzylammonium chloride, alkyltrimethylammonium chloride, octyltrimethylammonium chloride, dodecyltrimethylammonium chloride, benzalkonium chloride, and benzethonium chloride;
Alkylamine salt type surfactants such as monomethylamine hydrochloride and dimethylamine hydrochloride.
   Preferred examples of amphoteric surfactants include:
Amine oxide type surfactants such as lauryldimethylamine oxide, alkyl (C8-18) dimethylamine oxides (N,N-dimethylalkyl (C8-18) amine oxides), coconut alkyldimethylamine oxide, decyldimethylamine oxide, myristyldimethylamine oxide, dihydroxyethyllaurylamine oxide, and oleyl dimethylamine oxide;
Betaine type surfactants such as lauryldimethylaminoacetic acid betaine, laurylhydroxysulfobetaine, stearyldimethylaminoacetic acid betaine, dodecylaminomethyldimethylsulfopropyl betaine, coconut fatty acid (C8-18) amidopropyl betaine, 2-alkyl-N-carboxymethyl-N-hydroxyethylimidazolinium betaine, and lauramidopropyl betaine.
   These surfactants may be used alone or in combination of two or more.

In a formulation of the liquid composition intended for moisturizing, the content ratio of the surfactant in the liquid composition is preferably 0.05 to 40.0% by mass, more preferably 0.1 to 30% by mass, and still more preferably 0.3 to 20% by mass.

When the content ratio of the surfactant is within this range, the oil component described above can be uniformly dispersed when preparing an oil-in-water emulsion, and even when stored for a long period of time, separation of water and oil can be prevented, thereby maintaining a uniform mixed state.
In a formulation of the liquid composition intended for cleansing, the content ratio of the surfactant in the liquid composition is preferably 0.5 to 60% by mass, more preferably 1 to 55% by mass, and still more preferably 2 to 50% by mass. When the content ratio of the surfactant is within this range, cleansing power can be enhanced by allowing it to blend with dirt, rinsing can be made easier, and an excellent sensory characteristics upon use can be obtained.

Examples of thickeners that may be contained in the liquid composition include polysaccharides such as xanthan gum, guar gum, methylcellulose, ethylcellulose, and hydrophobically modified hydroxypropylmethylcellulose; carboxyvinyl polymer; copolymers of acrylic acid and alkyl methacrylate; and polymeric esters such as dextrin palmitate and inulin stearate.

These thickeners may be used alone or in combination of two or more. By means of these thickeners, when applied to the body of a human or the like, the composition does not run off the skin and can be spread easily.

The content ratio of the thickener in the liquid composition is preferably 0.05 to 7% by mass, more preferably 0.1 to 5% by mass. When the content ratio of the thickener is within this range, the composition can be prevented from running off the skin and can be spread easily.

By including such thickeners, the viscosity of the liquid composition can be adjusted to about 0 to 100,000 mPa·s at 25° C., preferably 0.5 to 80,000 mPa·s, and more preferably 3 to 60,000 mPa·s. When the viscosity of the liquid composition is within this range, bubbles can remain in the applicate for a longer period of time when the applicate containing bubbles is applied to the skin of a human or the like.

The liquid composition of the present invention may be a solubilized composition, a gel, or an emulsified composition.

A solubilized composition refers to one having a light transmittance at a wavelength of 660 nm of, for example, 50% or more, preferably 70% or more. When the composition is an emulsified composition, it is preferably an oil-in-water type emulsion, but may also be a water-in-oil type emulsion.

In one embodiment of the present invention, the ratio of the oil phase to the water phase is not limited, but is preferably about 1:0.5 to 99, more preferably about 1:5 to 80, and still more preferably about 1:10 to 50.

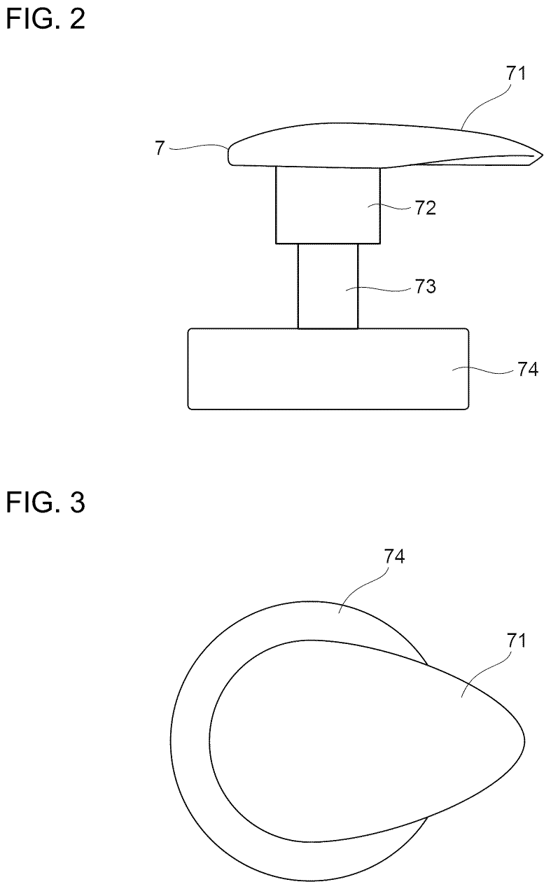

In this manner, when the liquid composition in which various compounds are mixed is applied from the tip of the nozzle portion 71 of the cap portion 7, which is indirectly provided on the container 1 via the mounting cup 3 provided at the opening of the container 1 and the stem 4 provided on the mounting cup 3, it is preferred that the applied applicate contain 1.8×10⁸ to 1.0×10¹⁴ bubbles/ml having diameters of 1 nm or more and less than 1000 nm, and more preferably 2.0×10⁸ to 5.0×10¹³ bubbles/ml. The cap portion 7 is provided with a nozzle and may have a structure that covers a part of the mounting cup. Further, the cap portion 7 may be a pump-type cap having the shape represented by the side views shown in FIGS. 2, 4, 6, and 8.

FIGS. 3, 5, 7, and 9 are top views corresponding to the side views of FIGS. 2, 4, 6, and 8, respectively.

In one embodiment of the aerosol product of the present invention, the material of the stem 4 and the housing used for the valve is preferably at least one selected from polyimide, polyacetal, and polybutylene terephthalate, and more preferably polyacetal and polybutylene terephthalate.

In one embodiment of the aerosol product of the present invention, the material of the packing and stem rubber used for the valve is preferably at least one selected from butyl rubber, nitrile rubber, styrene-butadiene rubber, hydrogenated nitrile rubber, and olefin-based elastomers, and more preferably nitrile rubber.

In one embodiment of the aerosol product of the present invention, the material of the spring used for the valve is preferably SUS304.

In one embodiment of the aerosol product of the present invention, the material of the pipe used for the valve is preferably at least one selected from low-density polyethylene and polypropylene, and more preferably low-density polyethylene.

In one embodiment of the aerosol product of the present invention, the formulation discharged from the aerosol product contains bubbles having diameters of 1 nm or more and less than 1000 nm at the time of discharge and/or at the time of skin application, and the amount is preferably 1.8×10⁸ to 1.0×10¹⁴ bubbles/ml, more preferably about 2.0×10⁸ to 5.0×10¹³ bubbles/ml. Such an amount of bubbles is a value measured using a NanoSight NS300 manufactured by Malvern Panalytical Ltd., which calculates the hydrodynamic diameter based on the Stokes-Einstein equation from scattered light when laser light is irradiated. The measurement was carried out after diluting the sample to a measurable concentration.

The non-flammable compressed gas G of the present invention is filled inside the container 1 together with the liquid composition, and is a gas in a state of pressure within the container 1 higher than atmospheric pressure. By filling the non-flammable compressed gas G, a large number of bubbles can be contained at the time of discharge and/or at the time of skin application. Furthermore, since the non-flammable compressed gas G may also be discharged to the outside together with the liquid composition when the cap portion 7 is pressed, if the gas is non-flammable compressed gas, even when applied together with the liquid composition, ignition and combustion due to fire do not occur, and excellent safety is ensured. In addition, by using a non-flammable compressed gas, the effects of the present invention can be exhibited. That is, a large number of bubbles on the nanometer scale are generated in the formulation discharged from the aerosol product. Preferred examples of the non-flammable compressed gas include nitrogen, carbon dioxide, nitrous oxide, helium, argon, and compressed air, with nitrogen being more preferred because it is less soluble in water. When compressed nitrogen is used as the non-flammable gas G, it is preferred that the nitrogen content be 99.9% or more, and more preferably 99.99% or more.

It is most preferred that the proportion of the non-flammable compressed gas in the gas contained in the aerosol product be 100%. However, a liquefied gas may also be included as long as the effects of the present invention are not impaired. The proportion of the liquefied gas relative to the liquid composition is preferably 10% by mass or less, more preferably 5% by mass or less, and still more preferably 1% by mass or less. It is also preferred that no liquefied gas be included at all. The lower limit of the liquefied gas may be about 0.1% by mass or about 0.2% by mass.

The pressure of the non-flammable compressed gas G filled in the container 1 together with the liquid composition is preferably 0.1 to 1.0 MPa at 25° C., more preferably 0.3 to 0.9 MPa. When the pressure of the non-flammable compressed gas G is within this range, the liquid composition can be smoothly discharged together with the non-flammable compressed gas G, and at the time of discharge and/or at the time of skin application, a large number of bubbles having diameters of about 1 nm or more and less than 1000 nm can be contained.

As a first embodiment, the aerosol product of the present invention, as shown in FIG. 1, comprises a container 1; a liquid composition contained in the container 1; a mounting cup 3 covering the opening of the container 1; a stem 4 inserted approximately in the center of the mounting cup 3 and having a discharge hole capable of discharging the liquid composition to the outside of the container 1 and a stem hole 41 communicating with the discharge hole and also communicating with the interior of the container 1; an elastic spring 5 that biases the stem 4 upward; a tube 6 inserted into the lower portion of the mounting cup 3 for guiding the liquid composition to the stem 4; and a cap portion 7 provided at the upper portion of the stem 4 and having an applicator port 71 communicating with the discharge hole of the stem 4. The liquid composition and the non-flammable compressed gas G contained in the container 1 are at a pressure higher than the outside of the container 1. Therefore, when the cap portion 7 is pressed, the stem 4 is also pressed against the biasing force of the elastic spring 5, whereby the stem hole 41 communicates with the interior of the container 1, and the liquid composition pressurized by the non-flammable compressed gas G is discharged to the outside from the applicator port 71 of the cap portion 7 through the tube 6 and the stem 4.

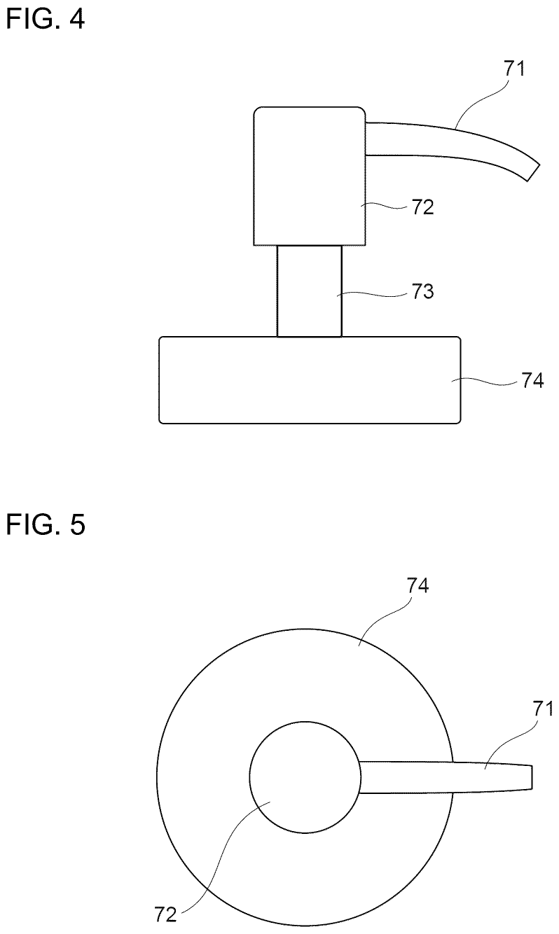

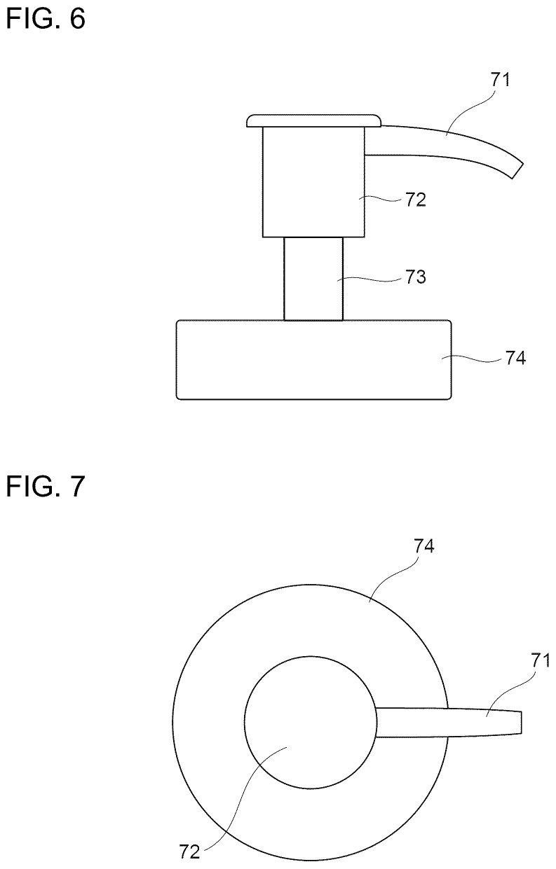

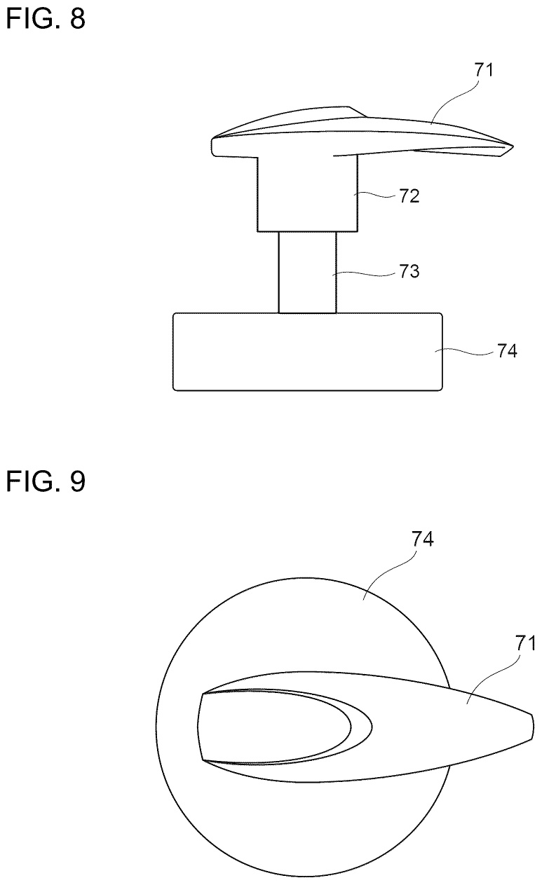

The shape of the nozzle portion 71 is not particularly limited. In one embodiment, as shown in FIG. 2, the nozzle 71 may have a shape integrated with a member covering the top of the joint portion 72 that is in contact with the nozzle 71. In another embodiment, as shown in FIG. 4 or FIG. 6, the nozzle 71 may have a structure joined to the joint portion 72. In still another embodiment, as shown in FIG. 8, the nozzle and the joint portion may be formed in an integrated structure.

In one embodiment, a cylinder 73 is in contact with the container and is fixed to a container covering portion 74 that directly covers the upper portion of the container, and the joint portion 72 is provided in a movable state on the outer periphery of the cylinder 73.

The nozzle portion may have a shape such as cylindrical, elliptical cylindrical, oblong cylindrical, or polygonal cylindrical.

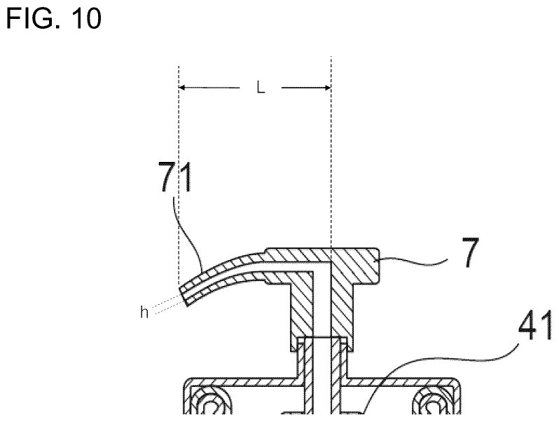

In one embodiment, the length of the nozzle portion (for example, the length indicated as L in FIG. 10) is preferably about 5 to 70 mm, more preferably about 10 to 65 mm, and still more preferably about 15 to 60 mm.

The shape of the opening (discharge portion) at the tip of the nozzle may be circular, elliptical, triangular, quadrilateral, polygonal, or any other shape. The area of the opening is preferably about 1.5 to 20 mm², more preferably about 3 to 15 mm².

In one embodiment, the inner diameter (maximum inner diameter; length indicated as h) of the opening (discharge portion) at the tip of the nozzle is preferably about 1 to 10 mm, more preferably about 1 to 8 mm, and still more preferably about 1.2 to 5 mm.

It is preferred that the ratio of the nozzle length L to the inner diameter of the opening at the nozzle tip be L/h=1 to 20, more preferably 1 to 15, and still more preferably 1 to 12.

When the nozzle portion has the above-described shape and size, it exhibits excellent discharge characteristics and is less likely to cause so-called dripping.

Furthermore, bubbles having diameters of 1 nm or more and less than 1000 nm are generated, and the bubbles are long-lasting even after being exposed to air, thereby promoting penetration of the liquid composition into the skin.

The aerosol product of the present invention produced in this manner preferably has a total volume of 5 to 1000 ml, more preferably 10 to 600 ml, and furthermore preferably has a weight of 10 to 800 g, more preferably 50 to 500 g. When the aerosol product of the present invention falls within these ranges, the user can carry and transport it, and it is particularly excellent in portability. In addition, in the aerosol product of the present invention, even if the liquid composition 2 does not contain many bubbles of nanometer order in diameter at the time of manufacture of the aerosol product, when applied for use on the skin, bubbles of nanometer order in diameter can be included in large amounts, for example by entraining air in the vicinity of the applicator port 71. The generation of such bubbles can also be identified by the phenomenon that, when left standing under reduced pressure, the liquid level rises compared to before the pressure reduction, and the volume increases after 90 seconds. That is, when discharged from the aerosol product, if a large number of bubbles of nanometer order in diameter are contained, then when subjected to reduced pressure (for example, −0.08 MPa, left standing for 1 minute), the gas in the bubbles expands, and as a result, the volume of the composition increases. Preferably, the presence of bubbles can be confirmed by the phenomenon that the volume increases 1.5 times or more, more preferably 1.8 times or more, and still more preferably 2 times or more. The expansion may be about 10 times or 100 times. Preferably, the expansion is 1.5 times or more and 100 times or less, and more preferably 1.8 times or more and 10 times or less.

### (Uses)

The aerosol product of the present invention is used as an oral care product, a cleansing product, a hair-related product, and/or a topical skin preparation, and is preferably used as a topical skin preparation. The topical skin preparations or hair-related products may include formulations intended for moisturizing or formulations intended for cleansing. Here, examples of oral care products include mouthwashes and toothpaste products; examples of cleansing products include, for example, car cleaners; examples of hair-related products include hair growth agents, hair tonics, hair sprays, and hair dyes; and examples of topical skin preparations include lotions, emulsions, cleansing agents, face washes, acne face washes, acne care agents, body soaps, serums, body creams, hand creams, formulations for treatment or prevention of dermatitis, antipruritics, anti-allergic agents, formulations for improving chapped hands, antibacterial agents, and antiviral agents.

### EXAMPLES

Hereinafter, the liquid compositions of the present invention will be described in detail.

It should be noted that the present invention is not limited to the following examples.

Liquid compositions having the formulations shown in Tables 1, 3, 4, and 5 were prepared, respectively.

In preparing the compositions of Examples 1-1, 1-5, 2-1, and 3-1, and Comparative Example 3-1, each component was mixed in a 200 ml Griffin beaker at a room temperature of 25° C., followed by addition of a viscosity modifier and mixing.

In preparing the compositions of Examples 1-2, 1-4, 1-7, 1-8, 2-2, 3-2, and 3-3, and Comparative Examples 2-1 and 3-2, the aqueous phase was mixed in a 200 ml Griffin beaker and the oil phase was mixed in another Griffin beaker at a room temperature of 25° C., followed by heating the aqueous and oil phases to about 80° C., mixing them, and cooling to about 40° C. while stirring.

In preparing the compositions of Examples 1-3, 1-6, 2-3, 2-4, and 2-5, and Comparative Example 1-1, each component was mixed in a 200 ml Griffin beaker at a room temperature of 25° C.

In preparing the compositions of Examples 4-1, 4-2, 4-3, 4-4, and 4-5, and Comparative Example 4-1, each component was mixed in a 200 ml Griffin beaker at a room temperature of 25° C., followed by heating to about 50° C. and stirring, and then cooling to about 25° C. while stirring.

The viscosity of each liquid composition was measured at 25° C. using a Brookfield-type rotational viscometer, Type B (manufactured by Tokimec Inc., trade name “TVB-10M,” Rotor No. 4), as described in JIS K7117-1.

The components, viscosity, pH, and other physical properties of the liquid compositions 2 of the Examples and Comparative Examples are shown in Tables 1, 3, and 4.

TABLE 1

| | Example | Example | Example | Example | Example | Example | Example | Example | Comparative |
|---|---|---|---|---|---|---|---|---|---|
| | 1-1 | 1-2 | 1-3 | 1-4 | 1-5 | 1-6 | 1-7 | 1-8 | Example 1-1 |
| Heparinoid | 0.1 | 0.1 | 0.1 | 0.3 | 0.3 | 0.3 | 0.1 | 0.1 | 0.1 |
| Dipotassium Glycyrrhizinate | 0.1 | 0.1 | 0.1 | — | — | — | 0.1 | 0.1 | 0.1 |
| Xanthan gum | 0.3 | 0.3 | — | 0.3 | 0.3 | — | 0.3 | 0.3 | — |
| Acrylic acid and methacrylic acid | 0.15 | 0.15 | — | — | — | — | 0.15 | 0.15 | — |
| alkyl copolymer | | | | | | | | | |
| Carbomer | — | — | — | 0.2 | 0.15 | — | — | — | — |
| Phenoxyethanol | 0.5 | 0.5 | 0.5 | 0.5 | 0.5 | 0.5 | 0.5 | 0.5 | 0.5 |
| L-Arginine | 0.08 | 0.08 | — | — | — | — | 0.08 | 0.08 | — |
| 1% aqueous sodium hydroxide solution | — | — | — | 5 | 0.08 | — | — | — | — |
| 1,3-BG | 5 | 6 | 5 | 6 | 5 | 5 | 6 | 6 | 5 |
| Glycerin | 10 | — | 10 | — | 10 | 10 | — | — | 10 |
| Vegetable squalane | — | 3 | — | — | — | — | 3 | 3 | — |
| Squalane | — | — | — | 3 | — | — | — | — | — |
| Olive oil | — | 1 | — | 1 | — | — | 1 | 1 | — |
| Dimethicone | — | 1 | — | 1 | — | — | 1 | 1 | — |
| Behenyl alcohol | — | 0.5 | — | 1 | — | — | 0.5 | 0.5 | — |
| Polyethylene glycol monostearate | — | 1 | — | 2 | — | — | 1 | 1 | — |
| Glyceryl monostearate | — | 0.5 | — | 0.5 | — | — | 0.5 | 0.5 | — |
| Purified water | q.s. to | q.s. to | q.s. to | q.s. to | q.s. to | q.s. to | q.s. to | q.s. to | q.s. to |
| | 100% | 100% | 100% | 100% | 100% | 100% | 100% | 100% | 100% |
| Total amount | 100 | 100 | 100 | 100 | 100 | 100 | 100 | 100 | 100 |
| Viscosity (mPa · s) | 2,360 | 4,580 | — | 3,880 | 1,840 | — | 4,580 | 4,580 | — |
| pH | 5.3 | 5.3 | 5.3 | 6.0 | 4.5 | 5.3 | 5.3 | 5.3 | 5.3 |
| Ratio of liquefied gas | — | — | — | — | — | — | 3 | 8 | — |
| Filling pressure | 0.8 | 0.8 | 0.8 | 0.8 | 0.8 | 0.8 | 0.8 | 0.8 | 0.8 |

(percentage by mass)

### Examples

The liquid compositions formulated as described above were placed into containers, to which the mounting cup 3, stem 4, and cap portion 7 were attached. Nitrogen or carbon dioxide was then charged through the stem 4 until the internal pressure of the container 1 reached 0.8 MPa at 25° C. (for the compositions in Table 1) or the pressures shown in the tables (for the compositions in Tables 3 to 5), thereby producing aerosol products as shown in FIG. 1.

### Comparative Examples

The liquid composition of Formulation Example 1 was placed into a predetermined container, and a finger-spray device, which discharges the contents by the force of a user's finger, was attached to the container to produce a finger-spray product. That is, in Comparative Example 1, no non-flammable compressed gas was filled, and only atmospheric air was present inside the container, such that when the finger-spray was pressed during use, the liquid composition contained inside was discharged from the discharge port. In addition, Comparative Example 2-1 was a test conducted using a similar aerosol product prepared in the Examples.

### Test Example 1: Viscosity Measurement

Using the aerosol products of the respective Examples to discharge the liquid compositions, and using the finger-spray product of Comparative Example 1 to discharge the liquid composition, the viscosities of the discharged materials were measured at 25° C. with a Brookfield-type rotational viscometer, Type B (manufactured by Tokimec Inc., trade name “TVB-10M”), employing SPINDLE No. M4 at 12 rpm, as described in JIS K7117-1.

### Test Example 2: pH Measurement

Using the aerosol products of the respective Examples to discharge the liquid compositions, and using the finger-spray product of Comparative Example 1 to discharge the liquid composition, the pH of the discharged materials was measured at a liquid temperature of 25° C. with a commercially available pH meter (for example, Model F-52 (registered trademark) manufactured by Horiba, Ltd.).

### Test Example 3: Measurement of Bubble Amount

Using the aerosol products of the respective Examples and Comparative Examples to discharge the liquid compositions, the amount of bubbles having diameters of 1 nm or more and less than 1000 nm was measured. For each aerosol product of the Examples and Comparative Examples, the amount of bubbles having diameters of 1 nm or more and less than 1000 nm, also referred to as ultrafine bubbles, contained in the applied material when the liquid composition 2 was discharged from the applicator port 71, was measured using a NanoSight NS300 manufactured by Malvern Panalytical Ltd.

Specifically, for each aerosol product of the Examples and Comparative Examples, the applied material discharged from the applicator port 71 was diluted with degassed water to a predetermined weight, the amount of bubbles was measured, and the measured bubble amount was multiplied by the dilution factor to convert it into the bubble amount in the applied material.

### Test Example 4: Sensory Evaluation Test

Using the aerosol products of the respective Examples and Comparative Examples, the discharged materials of the liquid compositions were tested for skin affinity when spread onto human skin.

### [Ease of Spreading]

At 25° C., for each aerosol product and spray product of the respective Examples and Comparative Examples, approximately 1 g of the liquid composition 2 was applied to the inner arm of a test subject and spread over the skin. The ease of spreading upon application was evaluated on a five-point scale, with “very easy to spread” as 5 points, “easy to spread” as 4 points, “normally easy to spread” as 3 points, “slightly difficult to spread” as 2 points, and “difficult to spread” as 1 point. The average score of five panelists was calculated. Products with an average score of 4 points or more were rated as ⊚, those with an average score of 3.5 points or more but less than 4 points were rated as ∘, those with an average score of 3.0 points or more but less than 3.5 points were rated as Δ, and those with an average score of less than 3.0 points were rated as x.

Among these, ⊚ and ∘ were judged as favorable (easy to spread), while Δ and x were judged as unfavorable (difficult to spread).

[Ease of Absorption into the Skin]

At 25° C., for each aerosol product and spray product of the respective Examples and Comparative Examples, approximately 1 g of the liquid composition 2 was applied to the inner arm of a test subject and spread over the skin. The ease of absorption into the skin upon application was evaluated on a five-point scale, with “very easily absorbed into the skin” as 5 points, “easily absorbed into the skin” as 4 points, “normally absorbed into the skin” as 3 points, “slightly difficult to absorb into the skin” as 2 points, and “difficult to absorb into the skin” as 1 point. The average score of five panelists was calculated. Products with an average score of 4 points or more were rated as ⊚, those with an average score of 3.5 points or more but less than 4 points were rated as ∘, those with an average score of 3.0 points or more but less than 3.5 points were rated as Δ, and those with an average score of less than 3.0 points were rated as x.

Among these, ⊚ and ∘ were judged as favorable (easily absorbed into the skin), while Δ and x were judged as unfavorable (difficult to absorb into the skin).

### [Moisturizing Effect]

At 25° C., for each aerosol product and spray product of the respective Examples and Comparative Examples, approximately 1 g of the liquid composition 2 was applied to the inner arm of a test subject and spread over the skin. The moisturizing effect upon application was evaluated on a five-point scale, with “moisturizing effect very well maintained” as 5 points, “moisturizing effect well maintained” as 4 points, “moisturizing effect normally maintained” as 3 points, “moisturizing effect poorly maintained” as 2 points, and “moisturizing effect not maintained” as 1 point. The average score of five panelists was calculated. Products with an average score of 4 points or more were rated as ⊚, those with an average score of 3.5 points or more but less than 4 points were rated as ∘, those with an average score of 3.0 points or more but less than 3.5 points were rated as Δ, and those with an average score of less than 3.0 points were rated as x.

Among these, ⊚ and ∘ were judged as favorable (moisturizing effect maintained), while Δ and x were judged as unfavorable (moisturizing effect not maintained).

### [Absence of Stickiness]

At 25° C., for each aerosol product and spray product of the respective Examples and Comparative Examples, approximately 1 g of the liquid composition 2 was applied to the inner arm of a test subject and spread over the skin. The absence of stickiness upon application was evaluated on a five-point scale, with “no stickiness at all” as 5 points, “no stickiness” as 4 points, “hardly any stickiness” as 3 points, “slight stickiness” as 2 points, and “very sticky” as 1 point. The average score of five panelists was calculated. Products with an average score of 4 points or more were rated as ⊚, those with an average score of 3.5 points or more but less than 4 points were rated as ∘, those with an average score of 3.0 points or more but less than 3.5 points were rated as Δ, and those with an average score of less than 3.0 points were rated as x.

Among these, ⊚ and ∘ were judged as favorable (less sticky), while Δ and x were judged as unfavorable (sticky).

In the evaluations of ease of spreading, ease of absorption into the skin, moisturizing effect, and absence of stickiness described above, products that were rated ⊚ or ∘ in all items were judged to be preferable because of their overall well-balanced performance.

The test results of these products of the Examples and Comparative Examples are shown in Table 2.

TABLE 2

| | | | | | | | | | Comparative |
|---|---|---|---|---|---|---|---|---|---|
| | Example1-1 | Example1-2 | Example1-3 | Example1-4 | Example1-5 | Example1-6 | Example1-7 | Example1-8 | Example1-1 |
| Nozzle length | 35 | 35 | 39 | 35 | 39 | 39 | 35 | 35 | 0 |
| L (mm) | | | | | | | | | |
| Inner diameter of | 3 | 3 | 4 | 3 | 4 | 4 | 3 | 3 | 0.2 |
| the nozzle opening | | | | | | | | | |
| (mm) | | | | | | | | | |
| Area of the nozzle | 7 | 7 | 13 | 7 | 13 | 13 | 7 | 7 | 0.03 |
| opening (mm²) | | | | | | | | | |
| Cap shape | circle | circle | ellipse | circle | ellipse | ellipse | circle | circle | circle |
| Evaluation | ⊚ | ⊚ | ⊚ | ⊚ | ⊚ | ⊚ | ⊚ | ⊚ | X |
| Ease of spreading | ⊚ | ⊚ | ⊚ | ⊚ | ⊚ | ⊚ | ⊚ | ⊚ | ◯ |
| Skin affinity | ⊚ | ⊚ | ⊚ | ⊚ | ⊚ | ⊚ | ⊚ | ⊚ | Δ |
| Moisturizing | ◯ | ⊚ | ◯ | ⊚ | ⊚ | ◯ | ⊚ | ⊚ | Δ |
| effect | | | | | | | | | |
| Stickiness | ⊚ | ⊚ | ⊚ | ◯ | ◯ | ⊚ | ⊚ | ⊚ | ◯ |
| Number of | 344 | 15100 | 7.4 | 389000 | 4470 | 6.2 | 2900 | 3000 | 0.9 |
| bubbles (×10⁸/mL) | | | | | | | | | |

As shown in Table 2, with respect to the aerosol products that comprise the liquid composition 2 containing water, polysaccharide moisturizing components, and compressed nitrogen as the non-flammable compressed gas G, and have an L/h ratio within the prescribed range, they were excellent in portability such that they could be carried and transported. Moreover, when used on the human body or the like, they could entrain air near the applicator port upon application, thereby including a large number of bubbles of nanometer order in diameter. Furthermore, when the applied material was spread on the skin, various performance characteristics were favorable, and the products were useful.

### Test Example 5: Discharge Performance Test

Using the aerosol products of the respective Examples and Comparative Examples, the liquid compositions were discharged. While the aerosol product was placed in a stationary position, the liquid composition was discharged for one second from the discharge port at a height of about 15 cm, and the discharge distance from the discharge port was confirmed.

### Evaluation

A discharge distance of 5 cm or more was rated ⊚, a discharge distance of 1 cm or more but less than 5 cm was rated ∘, and a discharge distance of less than 1 cm was rated x.

TABLE 3

| | Example | Example | Example | Example | Example | Comparative |
|---|---|---|---|---|---|---|
| | 2-1 | 2-2 | 2-3 | 2-4 | 2-5 | Example 2-1 |
| Heparinoid | 0.1 | 0.1 | 0.1 | 0.1 | 0.1 | 0.1 |
| Dipotassium Glycyrrhizinate | 0.1 | 0.1 | 0.1 | 0.1 | 0.1 | 0.1 |
| Xanthan gum | 0.3 | 0.3 | — | — | — | 0.3 |
| Acrylic acid and methacrylic | 0.15 | 0.15 | — | — | — | 0.15 |
| acid alkyl copolymer | | | | | | |
| Phenoxyethanol | 0.5 | 0.5 | 0.5 | 0.5 | 0.5 | 0.5 |
| L-Arginine | 0.08 | 0.08 | — | — | — | 0.08 |
| 1,3-BG | 5 | 6 | 5 | 5 | 5 | 6 |
| Glycerin | 10 | — | 10 | 10 | 10 | — |
| Vegetable squalane | — | 3 | — | — | — | 3 |
| Olive oil | — | 1 | — | — | — | 1 |
| Dimethicone | — | 1 | — | — | — | 1 |
| Behenyl alcohol | — | 0.5 | — | — | — | 0.5 |
| Polyethylene glycol | — | 1 | — | — | — | 1 |
| monostearate | | | | | | |
| Glyceryl monostearate | — | 0.5 | — | — | — | 0.5 |
| Purified water | q.s. to | q.s. to | q.s. to | q.s. to | q.s. to | q.s. to |
| | 100% | 100% | 100% | 100% | 100% | 100% |
| Total amount | 100 | 100 | 100 | 100 | 100 | 100 |
| Viscosity (mPa · s) | 2,360 | 4,580 | — | — | — | 2,360 |
| pH | 5.3 | 5.3 | 5.3 | 5.3 | 5.3 | 5.3 |
| Filling pressure | 0.8 | 0.8 | 0.8 | 0.6 | 0.3 | 0.8 |
| nozzle length L | 35 | 35 | 39 | 39 | 39 | 0 |
| Inner diameter of the nozzle | 3 | 3 | 4 | 4 | 4 | 0.2 |
| opening | | | | | | |
| Area of the nozzle opening | 7 | 7 | 13 | 13 | 13 | 0.03 |
| Discharge Performance | ◯ | ◯ | ⊚ | ⊚ | ◯ | X |
| | 1.6 cm | 1.5 cm | 9.3 cm | 7.6 cm | 3.0 cm | 0.1 cm |

As a result, it was confirmed that the aerosol products of the Examples exhibited favorable discharge distances during use and did not produce dripping.

### Test Example 6: Evaluation of Volume Change Under Reduced Pressure

The formulations discharged from the products of Examples 3-1, 3-2, and 3-3 were filled into 50 ml screw tubes (about φ35×85 mm) with scale to a height of about 1 cm. Similarly, the same liquid composition as that of Example 3-1 was filled into a non-gas pump container, discharged (Comparative Example 3-1), and filled into a graduated screw tube to a height of about 1 cm. The same procedure was performed for the compositions identical to those of Examples 3-2 and 3-3 (Comparative Example 3-2). Next, the screw tubes were placed in the center of a suction filtration bell jar (150φ×200H) manufactured by Sogo Rikagaku Glass Co., Ltd., and reduced pressure was applied to −0.08 MPa using a Nitto Kohki Linicon vacuum pump (LV-660-A2-0001). After being left standing for one minute, the volume change of each composition was confirmed. The test was carried out at a room temperature of 20° C.

TABLE 4

| | Example | Example | Example | Comparative | Comparative |
|---|---|---|---|---|---|
| | 3-1 | 3-2 | 3-3 | Example 3-1 | Example 3-2 |
| Heparinoid | 0.1 | 0.1 | 0.1 | 0.1 | 0.1 |
| Dipotassium Glycyrrhizinate | 0.1 | 0.1 | 0.1 | 0.1 | 0.1 |
| Xanthan gum | 0.3 | 0.3 | 0.3 | 0.3 | 0.3 |
| Acrylic acid and methacrylic | 0.15 | 0.15 | 0.15 | 0.15 | 0.15 |
| acid alkyl copolymer | | | | | |
| Phenoxyethanol | 0.5 | 0.5 | 0.5 | 0.5 | 0.5 |
| L-Arginine | 0.08 | 0.08 | 0.08 | 0.08 | 0.08 |
| 1,3-BG | 5 | 6 | 6 | 5 | 6 |
| Glycerin | 10 | — | — | 10 | — |
| Vegetable squalane | — | 3 | 3 | — | 3 |
| Olive oil | — | 1 | 1 | — | 1 |
| Dimethicone | — | 1 | 1 | — | 1 |
| Behenyl alcohol | — | 0.5 | 0.5 | — | 0.5 |
| Polyethylene glycol | — | 1 | 1 | — | 1 |
| monostearate | | | | | |
| Glyceryl monostearate | — | 0.5 | 0.5 | — | 0.5 |
| Purified water | q.s. to | q.s. to | q.s. to | q.s. to | q.s. to |
| | 100% | 100% | 100% | 100% | 100% |
| Total amount | 100 | 100 | 100 | 100 | 100 |
| Viscosity (mPa · s) | 2,360 | 4,580 | 4,580 | 2,360 | 4,580 |
| pH | 5.3 | 5.3 | 5.3 | 5.3 | 5.3 |
| Enclosed gas | nitrogen | nitrogen | carbon | — | — |
| | | | dioxide | | |
| Filling pressure | 0.8 | 0.8 | 0.55 | — | — |
| nozzle length L | 35 | 35 | 35 | 34 | 34 |
| Inner diameter of the nozzle | 3 | 3 | 3 | 4 | 4 |
| opening | | | | | |
| Area of the nozzle opening | 7 | 7 | 7 | 13 | 13 |
| Percentage | ◯ | ◯ | ◯ | X | X |
| increase in volume | 2.5 | 1.8 | 7 | 0 | 0 |
| under reduced pressure | | | | | |

### Test Example 7: Cleansing Performance Evaluation Test

A line about 5 cm long was drawn on a petri dish with eyeliner (Canmake Creamy Touch Liner, IDA Laboratories Co., Ltd.) and dried for 3 minutes. Similarly, concealer (Spotscover Foundation, Shiseido Co., Ltd.) was applied to another petri dish to a size of about 2×5 cm and dried for 3 minutes. The cleansing agents of the Examples and Comparative Examples were discharged and applied so as to cover the eyeliner line or the concealer area, respectively, and left standing for 3 minutes.

While rinsing with running water, the surface of the petri dish was rubbed 10 times clockwise. The cleansing function was evaluated by comparing with the initial state. The evaluation was made according to the following criteria, and the results are shown in Table 5.
⊚: The eyeliner or concealer was almost completely removed.
∘: More than half of the eyeliner or concealer was removed.
x: Hardly removed.

The lower of the two evaluations for eyeliner and concealer was taken as the overall evaluation, and products rated ∘ or higher were judged to be favorable.

TABLE 5

| | Example | Example | Example | Example | Example | Comparative |
|---|---|---|---|---|---|---|
| | 4-1 | 4-2 | 4-3 | 4-4 | 4-5 | Example 4-1 |
| Sorbitan tetraoleate | 10.8 | 10.8 | — | — | — | 10.8 |
| PEG-8 Distearate | 1.2 | 1.2 | — | — | — | 1.2 |
| PEG-8 Glyceryl | — | — | 14 | — | — | — |
| Isostearate | | | | | | |
| PEG-7 Glyceryl | — | — | 14 | — | — | — |
| Cocoate | | | | | | |
| PEG-15 Glyceryl | — | — | — | 29 | — | — |
| Isostearate | | | | | | |
| PEG-20 Glyceryl | — | — | — | — | 8 | — |
| Isostearate | | | | | | |
| PEG-20 Glyceryl | — | — | — | 7 | 20 | — |
| Triisostearate | | | | | | |
| Sorbitan | — | — | — | 3.2 | — | — |
| Sesquiisostearate | | | | | | |
| Mineral Oil | 27 | 27 | 14 | — | — | 27 |
| Triethylhexanoin | 25 | 25 | — | — | — | 25 |
| Ethylhexyl Palmitate | 25 | 25 | — | — | — | 25 |
| Cetyl Ethylhexanoate | 9 | 9 | 6 | — | — | 9 |
| Diethylhexyl Dimer | — | — | — | 8 | — | — |
| Dilinoleate | | | | | | |
| Glycerin | 1.0 | 1.0 | 16 | — | — | 1.0 |
| Diethylhexyl | — | — | — | 29.75 | — | — |
| Sebacate | | | | | | |
| 1,3-BGs | 0.1 | 0.1 | 5 | — | 2 | 0.1 |
| Dipropylene Glycol | — | — | — | 20 | 30 | — |
| Sodium Dilauroyl | 0.1 | 0.1 | — | — | — | 0.1 |
| Glutamate Lysine | | | | | | |
| Bis-Ethoxydiglycol | — | — | 2 | 2 | 2 | — |
| Cyclohexane 1,4- | | | | | | |
| Dicarboxylate | | | | | | |
| Polyglyceryl-2 | — | — | 1.5 | — | — | — |
| Diisostearate | | | | | | |
| Disodium EDTA | — | — | 0.1 | — | 0.1 | — |
| Citric Acid | — | — | 0.01 | — | 0.22 | — |
| Sodium Citrate | — | — | 0.09 | — | 0.1 | — |
| Tocopheryl Acetate | — | — | — | 0.05 | — | — |
| Phenoxyethanol | — | — | — | — | 0.5 | — |
| Purified water | q.s. to | q.s. to | q.s. to | — | q.s. to | q.s. to 100% |
| | 100% | 100% | 100% | | 100% | |
| Total amount | 100 | 100 | 100 | 100 | 100 | 100 |
| Viscosity (mPa · s) | 40 | 40 | 230 | 77 | 280 | 40 |
| Enclosed gas | nitrogen | carbon | nitrogen | nitrogen | nitrogen | — |
| | | dioxide | | | | |
| Filling pressure | 0.8 | 0.55 | 0.8 | 0.8 | 0.8 | — |
| nozzle length L | 35 | 35 | 35 | 35 | 35 | 34 |
| Inner diameter of | 3 | 3 | 3 | 3 | 3 | 4 |
| the nozzle opening | | | | | | |
| Area of the nozzle | 7 | 7 | 7 | 7 | 7 | 13 |
| opening | | | | | | |
| Cleansing | ⊚ | ⊚ | ⊚ | ⊚ | ◯ | ◯ |
| Performance | | | | | | |
| Evaluation Test | | | | | | |
| (eyeliner) | | | | | | |
| Cleansing | ⊚ | ◯ | ⊚ | ◯ | ◯ | X |
| Performance | | | | | | |
| Evaluation Test | | | | | | |
| (concealer) | | | | | | |
| Cleansing | ⊚ | ◯ | ⊚ | ◯ | ◯ | X |
| Performance | | | | | | |
| Evaluation Test | | | | | | |
| (comprehensive | | | | | | |
| evaluation) | | | | | | |

As a result, Examples 4-1 and 4-3 were able to almost completely remove the eyeliner and concealer, and Examples 4-2, 4-4, and 4-5 were able to remove more than half of the eyeliner and concealer, yielding favorable results.

Next, formulation examples are shown. In all of the formulation examples below, the mounting cup 3, stem 4, and cap portion 7 were attached, and nitrogen or carbon dioxide was filled through the stem 4 until the internal pressure of the container 1 at 25° C. reached the pressure shown in Table 6, thereby producing an aerosol product as shown in FIG. 1. The pump length L was 35 mm, and the diameter h of the nozzle discharge portion was 3 mm.

TABLE 6

| | Formulation | Formulation | Formulation | Formulation | Formulation | Formulation | Formulation |
|---|---|---|---|---|---|---|---|
| | Example 1 | Example 2 | Example 3 | Example 4 | Example 5 | Example 6 | Example 7 |
| Heparinoid | — | — | — | — | 0.1 | — | — |
| Dipotassium Glycyrrhizinate | 0.2 | — | — | — | — | — | — |
| Stearyl Glycyrrhetinate | — | — | — | 0.3 | — | — | — |
| Urea | — | — | — | — | — | — | 5 |
| Allantoin | 0.2 | — | — | — | 0.1 | — | — |
| Tocopheryl Acetate | — | — | 0.15 | 0.15 | — | — | — |
| Ascorbic Acid | — | 5 | — | — | — | 3 | — |
| Arbutin | — | — | — | — | — | 5 | — |
| Prunus Persica (Peach) Leaf | 0.1 | — | — | — | — | — | — |
| Extract | | | | | | | |
| ceramide3 | — | — | — | 0.3 | — | — | — |
| Xanthan gum | 0.3 | 0.3 | 0.3 | 0.3 | 0.3 | — | 0.3 |
| Acrylic acid and methacrylic | — | 0.15 | 0.15 | — | — | — | 0.15 |
| acid alkyl copolymer | | | | | | | |
| Carbomer | — | — | — | 0.2 | 0.15 | — | — |
| Phenoxyethanol | 0.5 | 0.5 | 0.5 | 0.5 | 0.5 | 0.5 | 0.5 |
| L-Arginine | — | 0.08 | 0.08 | — | 0.08 | — | 0.08 |
| 1% aqueous sodium hydroxide | — | — | — | 5 | — | — | — |
| solution | | | | | | | |
| 1,3-BG | 5 | 6 | 5 | 6 | 5 | 5 | 7 |
| Glycerin | 10 | — | 5 | — | 10 | 10 | 7 |
| Vegetable squalane | — | — | — | — | — | — | 3 |
| Squalane | — | 3 | 5 | 3 | — | — | — |
| Olive oil | — | 1 | — | 1 | 2 | — | — |
| Dimethicone | — | 1 | 1 | 1 | 0.2 | — | 0.2 |
| Behenyl alcohol | — | 1 | 1 | 1 | 0.5 | — | 1 |
| Polyethylene glycol | — | 1 | 1 | 2 | 2 | — | 2 |
| monostearate | | | | | | | |
| Glyceryl monostearate | — | 0.5 | 0.15 | 0.5 | 0.5 | — | 1 |
| Purified water | q.s. to | q.s. to | q.s. to | q.s. to | q.s. to | q.s. to | q.s. to |
| | 100% | 100% | 100% | 100% | 100% | 100% | 100% |
| Total amount | 100 | 100 | 100 | 100 | 100 | 100 | 100 |
| Filling pressure | 0.8 | 0.8 | 0.8 | 0.8 | 0.8 | 0.8 | 0.8 |

TABLE 7

| | Formulation | Formulation | Formulation | Formulation | Formulation | Formulation |
|---|---|---|---|---|---|---|
| | Example 8 | Example 9 | Example 10 | Example 11 | Example 12 | Example 13 |
| 3-O-ethyl ascorbic acid | 1 | — | — | — | — | — |
| tranexamic acid | — | 2 | — | — | — | — |
| Placenta extract | — | — | — | — | 2 | — |
| riboflavin | — | — | 0.01 | — | — | — |
| niacinamide | — | — | — | — | — | 0.2 |
| retinol | — | — | — | 0.1 | — | — |
| Hydroxypropyl Methylcellulose | 0.4 | — | — | — | — | — |
| Stearoxy Ether | | | | | | |
| ethanol | 7 | — | — | — | — | — |
| citric acid | 0.25 | — | — | — | — | — |
| sodium citrate | 0.2 | — | — | — | — | — |
| disodium edetate | 0.1 | — | — | — | — | — |
| Carbomer | — | 0.2 | 0.3 | 0.4 | — | — |
| Phenoxyethanol | 0.5 | 0.5 | 0.5 | 0.5 | 0.5 | 0.5 |
| Bis (2-ethoxyethanol) | 1.5 | — | — | — | — | — |
| cyclohexanecarboxylate | | | | | | |
| L-Arginine | — | 0.2 | 0.3 | 0.4 | — | — |
| 1,3-BG | 7 | 10 | — | — | 5 | 5 |
| Glycerin | 5 | 5 | 10 | 10 | 5 | 5 |
| Vegetable squalane | — | 5 | 2 | 7 | — | — |
| Olive oil | — | — | 5 | 2 | — | — |
| Dimethicone | — | 0.2 | 0.2 | 0.2 | — | — |
| Polyethylene glycol monostearate | — | 1 | 1 | 1 | — | — |
| Glyceryl monostearate | — | 1 | 1 | 1 | — | — |
| Purified water | q.s. to 100% | q.s. to 100% | q.s. to 100% | q.s. to 100% | q.s. to 100% | q.s. to 100% |
| Total amount | 100 | 100 | 100 | 100 | 100 | 100 |
| Filling pressure | 0.8 | 0.6 | 0.8 | 0.8 | 0.6 | 0.6 |

### DESCRIPTION OF REFERENCE SIGNS

1 . . . Container
2 . . . Liquid composition
3 . . . Mounting cup
4 . . . Stem
41 . . . Stem hole
5 . . . Elastic spring
6 . . . Tube
7 . . . Cap portion
71 . . . Applicator port
G . . . Non-flammable compressed gas
72 . . . Joint portion
73 . . . Cylindrical portion
74 . . . Container covering portion

1. An aerosol product comprising:
a container and a cap portion provided with a nozzle;
a liquid composition contained in the container; and
a non-flammable compressed gas filled in the container together with the liquid composition,
wherein an inner diameter h of a discharge portion of the nozzle and a nozzle length L satisfy L/h=1 to 20.

2. The aerosol product according to claim 1, wherein the non-flammable compressed gas is sealed at a pressure of 0.3 to 1.0 MPa at 25° C.

3. The aerosol product according to claim 1, wherein the viscosity of the liquid composition is 0 to 100,000 mPa's at 25° C.

4. The aerosol product according to claim 1, wherein the liquid composition is selected from the group consisting of a solubilized composition, a gel, and an emulsion.

5. The aerosol product according to claim 1, wherein the liquid composition comprises water or a polyhydric alcohol.

6. The aerosol product according to claim 1, wherein the liquid composition comprises at least one selected from the group consisting of glycyrrhizic acid or a salt thereof, glycyrrhetinic acid or a salt thereof, allantoin or a salt thereof, vitamin C group and derivatives thereof, tranexamic acid, arbutin, placenta, vitamin B group and derivatives thereof, vitamin E group and derivatives thereof, vitamin A group and derivatives thereof, urea, and polysaccharides.

7. The aerosol product according to claim 1, wherein the non-flammable compressed gas is nitrogen.

8. The aerosol product according to claim 1, wherein the liquid composition is a topical skin preparation, an oral care product, a cleansing product, or a hair-related product.
